(19) United States
(12) Patent Application Publication
(10) Pub. No.: US 2010/0144897 A1
(43) Pub. Date: Jun. 10, 2010

(54) Methicone replacements for cyclomethicone

(76) Inventors: Anthony J. O'Lenick, JR., Dacula, GA (US); Kevin Anthony O'Lenick, JR., Smyrna, GA (US)

Correspondence Address: A.J. O'Lenick, Jr., 2170 Luke Edwards Road, Dacula, GA, 30019, US

(21) Appl. No.: 12/378,052

(22) Filed: Feb. 11, 2009

Related U.S. Application Data
(60) Provisional application No. 61/201,089, filed on Dec. 8, 2008.

(51) Int. Cl. A61K 47/08; C07F 7/08

(52) U.S. Cl. 514/772; 556/456

(57) ABSTRACT

The invention is directed to a dry feeling silicone that not only provides a dry feel when applied to skin, but also has a low surface tension, provides spreadability and contains essentially no cyclic silicones and additionally and importantly cannot is not form to make cyclomethicone. The ability to make a product that (1) has the dry feel, (2) is cyclomethicone free, (3) is not capable of making cyclomethicone when exposed to catalyst and (4) is not flammable is a long felt need, unsatisfied need in the cosmetic industry.

### FEDERAL SPONSORSHIP

None

### FIELD OF THE INVENTION

The invention is directed to a dry feeling silicone that not only provides a dry feel when applied to skin, but also has a low surface tension, provides spreadability and contains essentially no cyclic silicones and additionally and importantly cannot is not form to make cyclomethicone.

### BACKGROUND OF THE INVENTION

There are a number of applications in which a dry feel is important. Cyclomethicones are commonly used in cosmetic products to provide a solvent that feels dry on the skin. Key areas where it is used include antiperspirants, color cosmetics and as a base solvent to blend with fragrance oils and perfume oils. Cyclomethicone is a clear, odorless silicone. It leaves a silky-smooth feel when applied to the skin. Cyclomethicones possess a cyclical structure rather than the chain structures of dimethyl silicones. Low heat of vaporization and the ability to select a desired vapor pressure has led their use as cosmetic vehicles. In other words the feel is associated with volatility. Volatility is the ability of the compound being tested to evaporate under the temperatures at which the compound is used in formulation. For cosmetic products, this temperature is ambient.

Prior to the present invention, it was generally accepted that cyclomethicones provided this feel because they evaporate quickly after helping to carry oils into the top layer of epidermis.

Cyclic silicones have been removed from more and more cosmetic formulations based upon health concerns. First the D4 was removed from cosmetic products, now D5 is under scrutiny. (D4 is octamethylcyclotetrasiloxane CAS 556-67-2 and D5 is cyclopentasiloxane (CAS #541-02-6)).

One of the simple approaches is to use linear low molecular weight silicone fluids as replacements for cyclomethicones (collectively D4 and D5 and mixtures thereof). Silicone fluids are a class of compounds that have become known by a variety of names including silicone oils, dimethyl fluids, dimethyl polysiloxane, and polydimethyl siloxane. Cyclic silicone compounds are used to make linear silicones. They are reacted with hexamethyldisloxane (commonly called MM) to make silicone fluids. The difficulty with this is the reaction results in an equilibration mixture of linear silicone and cyclics. The common process is to strip off the cyclics in the fluid prior to sale. Silicone fluids are synthesized by the equilibration reaction of MM and cyclomethicone.

The reaction may be run with either an acid or base catalyst. In one method, the reaction is conducted at room temperature for 12 hours, with sulfuric acid as a catalyst resulting in a mixture of linear fluid and cyclic silicone. If the catalyst is neutralized and the cyclic is stripped off, a stable fluid results. If the catalyst is not neutralized during stripping, the fluid will degrade back to cyclomethicone.

It is also quite interesting to note that a “finished silicone fluid” may be placed in contact with D4 and catalyst and re-equilibrated to make a higher viscosity fluid. Conversely, a “finished silicone fluid” may be re-equilibrated with MM and catalyst to make a lower viscosity fluid. Finally, silicone rubber may be decomposed into MM, and D4 via stripping of the product in the presence of catalyst. This property of silicone polymers makes them decidedly different from organic compounds.

It is exactly this property of equilibration, which occurs over a wide range of catalytic conditions that makes the linear fluids unacceptable as replacement for cyclomethicone. Simply put, it does not matter how well you strip the low molecular weight fluid, cyclomethicone will reform if it is exposed to conditions of catalysis favorable to its formation.

Other approaches like making blends on flammable organic solvents based upon polyisobutene and hydrogenated polyisobutene. The flammability and the effects of these materials on skin make them of little value as replacements for cyclomethicone.

The ability to make a product that (1) has the dry feel, (2) is cyclomethicone free, (3) is not capable of making cyclomethicone when exposed to catalyst and (4) is not flammable is a long felt need, unsatisfied need in the cosmetic industry.

### SUMMARY OF THE INVENTION

The invention is directed to a methicone polymer conforming to the following structure:

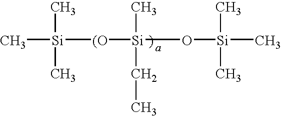

wherein:
a is an integer ranging from 1 to 6.

The invention is also directed to a process for providing conditioning and a dry feel to skin which comprises contacting the skin with an effective conditioning concentration of a methicone polymer conforming to the following structure:

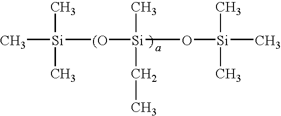

wherein:
a is an integer ranging from 1 to 6.

### DETAILED DESCRIPTION OF THE INVENTION

The ability to develop a product that meet the following requirements (1) has the dry feel, (2) is cyclomethicone free, (3) is not capable of making cyclomethicone when exposed to catalyst and (4) is not flammable required that we re-think what makes a material feel dry and how to develop a product that could meet the other requirements.

The first widely accepted concept that was incorrect and taught away from our invention is the widely held belief that dry feel comes only from volatility.

Since cosmetic products are applied at ambient temperature the volatility of products containing them likewise needs to be evaluate at ambient temperatures. In other words, if the product is used at room temperature the evaporation rate of the silicone at 100° C. is meaningless. Consumers will simply not use the product at that temperature for obvious reasons. To our surprise, we found that D4 is volatile under these test conditions loosing about half the weight in 7.5 hours, D5 used commonly as a replacement is not appreciably volatile at 20° C., retaining 95.5 percent of its weight after 7.5 hours. Linear silicones having a viscosity of 0.65 cps, 1 cps and 2 cps show very different volatility.

Volatility at 20° C.
% of starting material left

| | D5 | D4 | | | |
|---|---|---|---|---|---|
| Hours | Cyclic | Cyclic | 0.65 cps | 1 cps | 2 cps |
| 0.0 | 100.0% | 100.0% | 100.0% | 100.0% | 100.0% |
| 1.0 | 99.4% | 97.1% | 40.6% | 86.5% | 99.9% |
| 2.0 | 98.7% | 94.0% | 0.0% | 74.0% | 99.7% |
| 3.0 | 98.1% | 91.0% | 0.0% | 61.6% | 99.5% |
| 6.5 | 96.1% | 82.2% | 0.0% | 23.3% | 98.9% |
| 7.5 | 95.5% | 50.1% | 0.0% | 12.4% | 98.7% |
| 24 | 86.1% | 35.3% | 0.0% | 0.0% | 94.9% |

The above clearly shows that

(1) Silicones having a viscosity of 0.65 and 1 cst are volatile.

(2) D4 is more volatile than D5.

(3) Neither D5 nor 2 cst silicone fluid are appreciably volatile.

(4) Dry feel on skin at ambient temperature does not equate to volatility.

The next step was to design a molecule that has no possibility of degrading to give D4 or D5 during use. The approach here is to make a methicone not a dimethicone product.

Dimethicone compounds have two methyl groups around the internal Si. Compound 1 is a dimethicone ( dimethyl silicone).

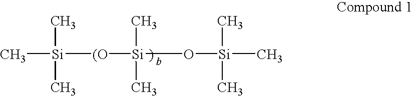

Dimethicone is made by the reaction of D4 and hexamethyl disiloxane also called MM.

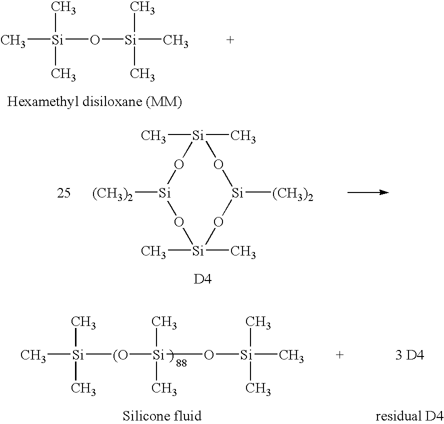

The residual D4 is stripped off, but it should be clear that the reaction is an equilibration reaction it can proceed in both directions.

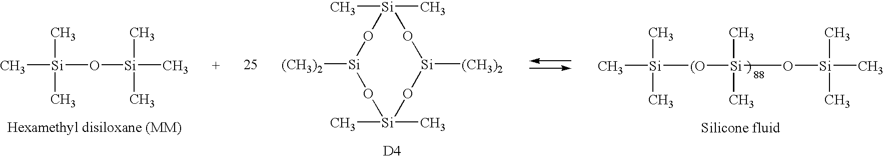

In contrast methicone compounds have only one methyl groups around the internal Si. Compound 2 is a methicone (methyl silicone).

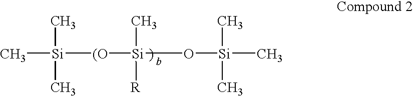

R is not CH₃

Methicones of the present invention are made by the reaction of silanic hydrogen polymers and ethylene. D4 is not present in the combined form (dimethicone) or in either reactant; consequently it cannot for on equilibration.

The key difference between methicones and dimethicones is important because it dictates the ability to re-form cyclic silicones. An article entitled Equilibration of Silicone Fluid written by O'Lenick et al and published in Cosmetics and Toiletries in May 2004 Vol. 119 No 5 page 89-98, clearly shows that silicone fluids will re-equilibrate to form cyclic compounds. The reaction is shown below:

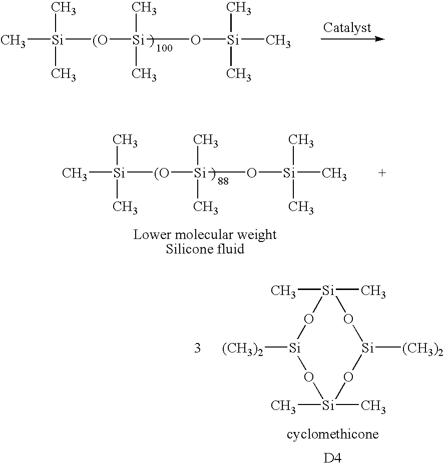

The compounds of the present invention have bulkier ethyl rather than ethyl groups present and do not recyclize. They also have a very low “a” value making cyclization improbable.

Keeping the value of “a” in the specified range and the internal substitution ethyl rather than methyl, result in a product that meets all the requirements. The product is not volatile, but has a dry feel because of its spreadability. This spreadability is due to low surface tension (22 dynes/cm²) and low viscosity.

Surprisingly and contrary to what one skilled in the art would predict these non-volatile materials meet all the requirements (1) has the dry feel, (2) is cyclomethicone free, (3) is not capable of making cyclomethicone when exposed to catalyst and (4) is not flammable. This is due to low surface tension (22 dynes/cm²) and low viscosity.

### PREPARATION OF THE COMPOUNDS OF THE PRESENT INVENTION

The compounds of the present invention are prepared by the reaction of a silanic hydrogen compound and ethylene. The silanic hydrogen compound used as raw material is items of commerce available form Siltech LLC Dacula, Ga. They conform to the following structure:

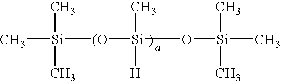

a is an integer ranging from 1 to 6.

### EXAMPLE

| Example | a |
|---|---|
| 1 | 1 |
| 2 | 4 |
| 3 | 6 |

Hydrosilylation with Ethylene

Ethylene is reacted with the silanic hydrogen compound in the presence of a catalyst selected from the group consisting of chloroplatinic acid and karnstedt catalyst. The vessel is pressurized and heated to 80° C. Ethylene is added under pressure and reacts as follows:

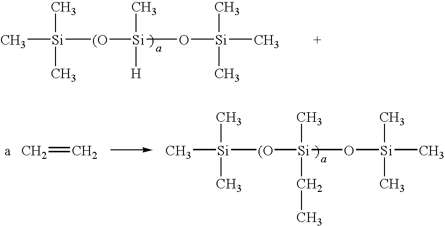

The ethylene pressure is maintained until the silanic hydrogen compound has ceased consuming ethylene.

Chloroplatinic acid is commercially available and has the formula H₂PtCl₆.

Karstedt catalyst is also commercially available and has the formula

Pt₂{[(CH₂═CH)Me₂Si]₂O}₃

Procedure

100 grams' of silanic hydrogen compound (example 1-3) and 500 ppm of Karnstedt catalyst are added to an autoclave, with good mixing. The autoclave is closed and heated to 80 C. Ethylene is then added under pressure. As the ethylene is consumed the pressure drops. The pressure is maintained during the reaction. Once all the ethylene reacts the pressure ceases to drop. That amount of ethylene is recorded below. The mixture is cooled and released to atmospheric.

| | Silanic Hydrogen | |
|---|---|---|
| Example | Example | Ethylene Added |
| 4 | 1 | 17.8 grams |
| 5 | 2 | 30.5 grams |
| 6 | 3 | 33.0 grams |

Applications Testing

The dryness was evaluated against D4 and D5 by consumer panel. The consumer was asked to apply 1 ml of compound to their left forearm and rub the material over forearm with their right index finger. They were to give D4 a value of 5 and rate the compounds between 1 and 5. (5 is most dry 1 is least).

| | Dry Feel |
|---|---|
| D4 | 5 |
| D5 | 3 |
| 1 Cst | 4 |
| 2 Cst | 2 |
| Example | 4 |

1. A methicone polymer conforming to the following structure: 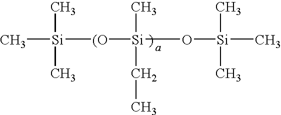
wherein:
a is an integer ranging from 1 to 6.

2. A methicone polymer of claim 1 wherein a is 1.

3. A methicone polymer of claim 1 wherein a is 4.

4. A methicone polymer of claim 1 wherein a is 6.

5. A process for conditioning skin which comprises contacting the skin with an effective conditioning concentration of a methicone polymer conforming to the following structure: 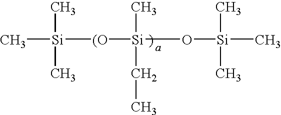
wherein:
a is an integer ranging from 1 to 6.

6. A process of claim 5 wherein said effective conditioning concentration ranges from 0.1 to 25% by weight.

7. A process of claim 5 wherein said effective conditioning concentration ranges from 0.5 to 10% by weight.

8. A process of claim 5 wherein a is 1.

9. A process of claim 5 wherein a is 4.

10. A process of claim 5 wherein a is 6.

11. A process of claim 6 wherein a is 1.

12. A process of claim 6 wherein a is 4.

13. A process of claim 6 wherein a is 6.

14. A process of claim 7 wherein a is 1.

15. A process of claim 7 wherein a is 4.

16. A process of claim 7 wherein a is 6.
